(19) United States
(12) Patent Application Publication
(10) Pub. No.: US 2018/0116645 A1
(43) Pub. Date: May 3, 2018

(54) Stylet and Needle Combinations Used to Collect Tissue Samples During Endoscopic Procedures

(71) Applicant: Michael NOSLER, Fort Collins, CO (US)

(72) Inventor: Michael NOSLER, Fort Collins, CO (US)

(21) Appl. No.: 15/568,023

(22) PCT Filed: Apr. 28, 2016
(86) PCT No.: PCT/US2016/029694

§ 371 Date: Oct. 20, 2017

Related U.S. Application Data
(60) Provisional application No. 62/153,609, filed on Apr. 28, 2015.

(51) Int. Cl. A61B 10/04; A61B 10/02

(52) U.S. Cl. CPC A61B 10/04; A61B 2010/045; A61B 10/0283

(57) ABSTRACT

A stylet formed with an extension having a shape adapted to collect a diagnostic sample of a target tissue and a method of using the same. A distal end of the stylet can be advanced axially beyond a distal end of a biopsy needle. Optionally, the stylet distal end bends radially at least partially beyond an exterior surface of the biopsy needle. At least a portion of the stylet may be sharpened to cut the target tissue. The stylet may also include void to collect and retain samples of the target tissue.

### CROSS-REFERENCE TO RELATED APPLICATIONS

This application claims priority under 35 U.S.C. § 119(e) to U.S. Provisional Patent Application Ser. No. 62/153,609 filed Apr. 28, 2015, which is incorporated herein in its entirety by reference.

### FIELD

The present disclosure relates to a biopsy device used to improve the collection of tissue samples during therapeutic and diagnostic endoscopy and endoscopic ultrasound.

### BACKGROUND

Needle biopsy procedures are common for the diagnosis and the staging of disease. In particular, during endoscopic ultrasound-guided fine needle aspiration (EUS-FNA), the needle is advanced under ultrasound guidance so that a physician using the EUS-FNA device is able to visualize a position of a needle in relation to a target tissue. Thus, EUS-FNA ensures that the correct tissue is sampled while minimizing risk to the patient. Endoscopes and needles used in EUS-FNA procedures are generally known and are described in U.S. Pat. No. 7,722,549, U.S. Design Pat. Nos. D657,461 and D690,009, and U.S. Patent Application Publication Nos. 2005/0090765, 2011/0046512, 2012/0116248, 2012/0197157, 2012/0226101, 2012/0253228, and 2012/0035500, each of which is hereby incorporated by reference, in its entirety.

Although EUS-FNA is a highly sensitive and specific procedure, it is often difficult to acquire a suitable tissue sample. Each EUS-FNA procedure often requires many attempts or passes to collect a tissue sample sufficient for diagnosis. Presently available needles and stylets used for the procedure are frequently designed to puncture or cut the target tissue but are occasionally ineffective in collecting samples. Most, if not all, known stylets have blunt tips that do not effectively cut samples of target tissue.

A known needle 112 with a stylet 116 positioned within a lumen of the needle is illustrated in FIG. 1. The needle 112 may pierce the target tissue 110 of a patient. However, the needle 112 frequently does not sever tissue from the target tissue 110. Accordingly, the needle 112 creates a “dry tap” or needle pass 114 that is acellular in which the needle 112 does not separate any cells or tissue from the target tissue 110. In this case, the needle 112 merely creates a hole 114, 114A, 114B in or through the target tissue 110. When no cells are separated from the target tissue 10, no cells can be aspirated through the needle 112 for analysis and diagnosis.

Existing needles 112 and stylets 116 commonly collect an insufficient number of cells of the target tissue 110 in each pass, or collect cells which are not diagnostic. Often, the only cells collected are blood cells or normal tissue cells collected as the needle 112 passes through the patient's body to the target tissue 110. Frequently, known needles 112 and stylets 116 only recover individual cells groups. Such samples make identification of the source of the cells difficult and complicate diagnosis. In these cases, resampling of the target tissue 110 is required, which may involve multiple instances of removal and insertion of a stylet 116 and the needle 112, which in turn requires removing and replacing a syringe coupled to the endoscope. Each additional pass 114, 114A, 114B increases the risk of contamination entering the surgical site and the time the patient is under anesthesia. The procedure is potentially traumatic because of the multiple needle passes that it necessitates.

For some EUS-FNA procedures, it may take up to ten needle passes to collect an adequate tissue sample that allows reliable diagnosis. For example, in a EUS-FNA demonstration observed by the inventor, the surgeon performing the procedure required fifteen needle passes to collect a tissue sample suitable for diagnosis. As it generally takes a minimum of about 5-7 minutes to test each sample, this means the patient was under anesthesia for more than about 1.3 hours to collect only one tissue sample.

Some needles and stylets have been developed to address these problems. However, some of these needles and stylets are difficult to work with or actuate during surgical procedures. Other attempts to overcome the deficiencies of known needles and stylets include methods of using the known needles and stylets. One such method developed to increase the amount of target tissue collected during a pass entails passing the needle through the target tissue and moving the needle back and forth, or “fanning” the needle, multiple times while suction is applied to collect a tissue sample. In some instances, as the needle is fanned in the target tissue, the end of the needle may move out of the target tissue resulting in a contaminated tissue sample. When fanning the needle, it is also difficult to tell if the needle is moving to different portions of the target tissue, as intended. Frequently, the fanning merely results in moving the needle back and forth within one hole formed in the target tissue. Further, some target tissues and tumors are fibrotic or calcified and fanning the needle simply does not work.

Accordingly, there is a need for an improved stylet and needle assembly that is easy to use in endoscopic procedures and is adapted to collect a diagnostic tissue sample with only one or minimal passes.

### SUMMARY

Embodiments of the present disclosure provide a stylet adapted to collect a tissue sample. The stylet is slidably-received within a lumen of a needle. One aspect of the present disclosure is to provide a stylet that can extend axially a predetermined distance beyond a distal end of the needle. In one example embodiment, the stylet can extend at least 1 mm beyond the needle distal end. Optionally, the stylet may be adapted to bend axially a predetermined amount when the stylet is at least partially extended from the needle. In an example embodiment, the stylet may bend axially at least 1 mm beyond an exterior surface of the needle.

Another aspect is to provide a cutting surface on a predetermined portion of the stylet. For example, the stylet may include sharpened edges that can cut and/or scrape target tissue to collect cells sufficient for analysis/diagnosis.

Another aspect of the present disclosure provides a stylet for EUS-FNA procedures that collects a greater quantity of cellular material from the target tissue than known stylet/needle assemblies. The stylet may include a recess or open chamber to collect the target tissue. In example embodiments, at least one edge proximate to a void has a hook shape such that as the stylet is withdrawn proximally, tissue is severed from the target tissue and collected within the associated void. Accordingly, the average size of each tissue sample collected by stylets of the present disclosure is generally larger than tissue samples collected by known stylets and needles.

The stylets of the present disclosure have many benefits for manufacturers, surgeons, and patients. For example, they may be used with known endoscopes and needles without modification. The stylets also improve the collection of tissue samples, thereby decreasing the amount of time required for collecting a diagnostic sample of a target tissue and reducing the time patients must remain on anesthesia. As a result, the stylets reduce the risk of the EUS-FNA procedure to the patient. These and other advantages will be apparent from the disclosure of the embodiments contained herein.

In example embodiments, the stylet is made at least partially of Nitinol. Because the stylet may be made of Nitinol, it has shape memory and can be formed into and hold a shape better than stylets made of other materials, thereby increasing the collection of tissue samples.

Thus, the present disclosure provides stylets for insertion through a lumen of a biopsy needle into a body of a patient along a tortuous path. In example embodiments, the stylets include an elongate body. A distal portion of the stylet extends longitudinally from a proximal portion. An extension is formed at the distal portion of the elongate body. Advancement of the stylet relative to the needle moves the extension from a retracted configuration at least partially in the lumen of the needle to an extended configuration in which the extension extends distally past a distal end of the biopsy needle. In one embodiment, the extension may project at least 2 mm past the distal end of the biopsy needle. The extension may also extend radially past a circumference of the body of the biopsy needle. In example embodiments, in the extended configuration, the extension projects up to about 6 mm past the distal end of the biopsy needle and up to about 6 mm radially past the circumference of the body of the biopsy needle. In example embodiments, the extension projects between about 2 mm and about 6 mm past the distal end of the biopsy needle. The extension of these embodiments may have a shape such as a hook, a spoon, a blade, and a point. The extension of these embodiments may include one or more sharp edges formed on the extension. The sharp edges are sufficiently sharp to cut tissue from a target area of a patient.

In certain embodiments, the stylet is configured to capture tissue from the target area of the patient. The stylet may include a tissue receiving cavity formed in a portion of the extension. The stylet of these embodiments may include one or more cavities formed on the extension. The cavity may communicate with a lumen formed in at least a portion of the stylet. In this manner, tissue samples collected by the stylet may be aspirated by applying suction without the necessity of removing the stylet from a lumen of the needle.

In example embodiments, the extension of the stylet is formed of Nitinol. In these embodiments, the critical temperature of the Nitinol may be selected to be less than a temperature in an operative environment for the stylet. In this manner, the desired shape for the extension is memorized for temperatures above the critical temperature so that the desired shape of the extension is restored during use when the stylet is in the extended configuration. In certain embodiments, the critical temperature is selected to be less than a body temperature of the patient. In related embodiments, in an extended configuration, an angle between the stylet and the extension is between about 10° and about 135°.

In example embodiments, at least a portion of the stylet is formed of one of stainless steel, copper, brass, aluminum, titanium, and combinations thereof.

In example embodiments, the stylet has a size and shape adapted to occupy substantially the entire cross-sectional area of the distal opening of the biopsy needle. In example embodiments, the biopsy needle is one of a 19 gauge, a 22 gauge, and a 25 gauge biopsy needle and the stylet has an exterior diameter adapted to slidably fit within the lumen of the biopsy needle.

It is one aspect of the present disclosure to provide a stylet for insertion through a lumen of a biopsy needle into a body of a patient. The stylet includes, but is not limited to: (1) an elongate body including a distal portion extending longitudinally from a proximal portion; and (2) an extension formed at the distal portion of the elongate body such that distal advancement of the stylet relative to the needle moves the extension from a retracted configuration in the lumen of the needle to an extended configuration in which the extension extends distally past a distal end of the biopsy needle. Optionally, in one embodiment, in the extended configuration, the extension projects up to about 6 mm past the distal end of the biopsy needle.

Additionally, one or more sharp edges may optionally be formed on the extension. The stylet may optionally include a tissue receiving cavity formed in the extension. In an embodiment, the tissue receiving cavity has a shape selected from a hook, a spoon, a blade, and a point.

In one embodiment, the biopsy needle is one of a 19 gauge, a 22 gauge, and a 25 gauge biopsy needle. The stylet has an exterior diameter adapted to slidably fit within the lumen of the biopsy needle. Optionally, the stylet has a size and shape adapted to occupy substantially an entire cross-sectional area of a distal opening of the biopsy needle.

In another embodiment, the extension is adapted to extend radially past a circumference of a body of the biopsy needle. A distal end of the extension may optionally be configured to extend up to about 6 mm radially past the circumference of the body of the biopsy needle. In another embodiment, in the extended configuration, an angle between the stylet and the extension is between about 10° and about 135°.

In some embodiments, at least a portion of the stylet is formed of one of stainless steel, copper, brass, aluminum, titanium, and combinations thereof. Additionally or alternatively, at least the extension of the stylet may be formed of Nitinol. In one embodiment, a critical temperature of the Nitinol is selected to be less than a temperature in an operative environment for the stylet. Optionally, a desired shape for the extension is memorized for temperatures above the critical temperature such that the desired shape of the extension is restored during use when the stylet is in the extended configuration. In another embodiment, the critical temperature is selected to be less than body temperature of the patient.

The stylet may be used to collect a sample of a target tissue of a patient. In another embodiment, the stylet may be used to reduce the amount of time required to collect a diagnostic sample of a target tissue of a patient. The stylet is also configured to increase an average size of a tissue sample collected from a target tissue of the patient.

Another aspect of the present disclosure is a method of using a stylet to collect a sample of a target tissue of a patient. The method includes, but is not limited to: (1) inserting a biopsy needle through the patient's body into the target tissue; (2) extending an extension of the stylet distally beyond an opening of the biopsy needle into the target tissue; (3) moving the extension of the stylet with respect to the target tissue; (4) withdrawing the stylet at least partially into a lumen of the biopsy needle; and (5) collecting a sample of target tissue of the patient from at least one of the patient's body, the biopsy needle, the surface of the stylet, and the lumen of the biopsy needle.

In one embodiment, in the extended configuration, the extension projects up to about 6 mm past the distal end of the biopsy needle. Additionally or alternatively, the extension of the stylet may be adapted to bend a predetermined amount radially when the extension is moved distally at least partially beyond the opening of the biopsy needle. For example, in an embodiment, a distal end of the extension extends up to about 6 mm radially past the circumference of the body of the biopsy needle. Additionally, in the extended configuration, an angle between the stylet and the extension may be between about 10° and about 135°.

The stylet may optionally include at least one of a sharp edge and a cavity to receive tissue. In embodiments, the tissue receiving cavity has a shape selected from a hook, a spoon, a blade, and a point.

In one embodiment, moving the extension with respect to the target tissue comprises at least one of: rotating the stylet axially; and moving the stylet proximally and distally.

At least a portion of the stylet may be formed of one of stainless steel, copper, brass, aluminum, titanium, and combinations thereof. Optionally, at least the extension of the stylet is formed at least partially of Nitinol. In some embodiments, the stylet has an exterior diameter adapted to slidably fit within the lumen of the biopsy needle that is one of a 19 gauge, a 22 gauge, and a 25 gauge biopsy needle. Additionally, the stylet may have a size and a shape configured to occupy substantially an entire cross-sectional area of a distal opening of the biopsy needle.

Yet another aspect of the present disclosure is to provide a device for collecting a sample of target tissue of a patient. The device generally comprises: (1) an actuator subassembly including a first lumen; (2) a needle including a second lumen with a distal opening, a portion of the needle positioned within the first lumen; and (3) a stylet positioned at least partially within the second lumen, the stylet comprising an extension with an edge adapted to cut the target tissue, the extension operable to extend a predetermined distance beyond the distal opening of the needle. Optionally, at least a portion of the stylet is formed of one or more of stainless steel, copper, brass, aluminum, titanium, Nitinol, and combinations thereof.

In one embodiment, the stylet further comprises a tissue receiving cavity formed in the extension. Optionally, at least the tissue receiving cavity of the extension is operable to extend beyond the distal opening of the needle. In one embodiment, the extension projects up to about 6 mm past the distal end of the biopsy needle. Additionally or alternatively, a distal end of the extension extends up to about 6 mm radially past the circumference of the body of the biopsy needle. Accordingly, in one embodiment, in the extended configuration an angle between the stylet and the extension is between about 10° and about 135°.

The stylet may be configured to slidably fit within the lumen of a biopsy needle of any predetermined gauge. In one embodiment, the biopsy needle is one of a 19 gauge, a 22 gauge, and a 25 gauge biopsy needle. Additionally, a size and shape of the stylet may be adapted to occupy substantially an entire cross-sectional area of the distal opening of the biopsy needle.

The actuator subassembly may further comprise a stylet actuator to move the stylet from a retracted position to an extended position and to rotate the stylet axially. Optionally, the stylet actuator may include at least one projection to indicate a position and an orientation of the extension of the stylet in relation to the distal opening of the needle.

The device may be used to collect a sample of a target tissue of a patient. In another embodiment, the device may be used to reduce the amount of time required to collect a diagnostic sample of a target tissue of a patient. The device is also configured to increase an average size of a tissue sample collected from a target tissue of the patient.

Another aspect of this disclosure provides a device configured to increase an average size of a tissue sample collected from a target tissue of a subject. The device includes, but is not limited to: (1) an actuator subassembly including a first lumen; (2) a needle including a second lumen with a distal opening, a portion of the needle positioned within the first lumen; and (3) a stylet positioned at least partially within the second lumen, the stylet comprising an extension with an edge adapted to cut the target tissue, the extension operable to extend a predetermined distance beyond the distal opening of the needle. Optionally, at least a portion of the stylet is formed of one or more of stainless steel, copper, brass, aluminum, titanium, Nitinol, and combinations thereof. Additionally, a plurality of sharp edges may be formed on the stylet.

The stylet may further comprise a tissue receiving cavity formed in the extension. Optionally, at least the tissue receiving cavity of the extension is operable to extend beyond the distal opening of the needle. In one embodiment, the extension projects up to about 6 mm past the distal end of the biopsy needle.

Additionally or alternatively, a distal end of the extension may extend up to about 6 mm radially past the circumference of the body of the biopsy needle. In an example embodiment, in the extended configuration the angle between the stylet and the extension is between about 10° and about 135°.

The stylet may be configured to slidably fit within the lumen of a biopsy needle of any predetermined gauge. The biopsy needle may be a 19 gauge, a 22 gauge, or a 25 gauge biopsy needle. Additionally, the size and shape of the stylet may be adapted to occupy substantially an entire cross-sectional area of the distal opening of the biopsy needle.

The actuator subassembly may further comprise a stylet actuator to move the stylet from a retracted position to an extended position and to rotate the stylet axially. Optionally, the stylet actuator may include at least one projection to indicate a position and an orientation of the extension of the stylet in relation to the distal opening of the needle.

This disclosure also provides a method of using a novel stylet to collect a sample of a target tissue of a patient. The method includes, but is not limited to: (1) localizing and delineating the target tissue in the patient; (2) inserting a biopsy needle with a lumen through the patient's body proximate to the target tissue; (3) extending an extension of the stylet positioned within the lumen into an extended position; (4) moving the extension with respect to the target tissue; (5) withdrawing the stylet, at least partially, into the lumen of the biopsy needle; and, (6) collecting a sample of target tissue of the patient from at least one of the patient's body, the biopsy needle, the surface of the stylet, and the lumen of the biopsy needle.

These and other advantages will be apparent from this disclosure. The above-described embodiments, objectives, and configurations are neither complete nor exhaustive. As will be appreciated, other embodiments of the invention are possible using, alone or in combination, one or more of the features set forth above or described below. Further, the Summary is neither intended nor should it be construed as representing the full extent and scope of the present disclosure. The invention is set forth in various levels of detail in the Summary, and, in the attached drawings and the Detailed Description and no limitation as to the scope of the invention is intended to either the inclusion or non-inclusion of elements, components, etc. in this Summary. Additional aspects of the invention will become more readily apparent from the detailed description, particularly when taken with the drawings.

References made herein to “advanced therapeutic endoscopy,” “endoscopic ultrasound,” “EUS-FNA procedures,” and “endoscopic ultrasound fine needle aspiration” or aspects thereof should not necessarily be construed as limiting the disclosure to a particular method of collecting tissue samples. It will be recognized by one skilled in the art that the invention may be used in any procedure and with any device of this disclosure for collecting tissue samples of any type.

The use of the term “distal” herein refers to a direction away from a user, such as a physician, and toward a target tissue area.

The term “proximal” refers to a direction approaching a user of the device (e.g., a physician) with a proximal portion of the device remaining external to the patient as the distal portion is inserted into the body of the patient.

Unless otherwise indicated, all numbers expressing quantities, dimensions, conditions, and so forth used in the specification and claims are to be understood as being modified in all instances by the term “about.”

The term “a” or “an” entity, as used herein, refers to one or more of that entity. As such, the terms “a” (or “an”), “one or more,” and “at least one” can be used interchangeably herein.

The use of “including,” “comprising,” or “having” and variations thereof herein is meant to encompass the items listed thereafter and equivalents thereof as well as additional items. Accordingly, the terms “including,” “comprising,” or “having” and variations thereof can be used interchangeably herein.

### BRIEF DESCRIPTION OF THE DRAWINGS

The accompanying drawings, which are incorporated in and constitute a part of the disclosure, illustrate embodiments of the disclosure and together with the Summary given above and the Detailed Description of the drawings given below, serve to explain the principles of these embodiments. In certain instances, details that are not necessary for an understanding of the disclosure may have been omitted. It should be understood, of course, that the disclosure is not necessarily limited to the particular embodiments illustrated herein. Additionally, it should be understood that the drawings are not necessarily to scale.

FIG. 1 is a schematic view of a known needle inserted into a target tissue of a patient, the needle includes a stylet within a lumen of the needle;

FIG. 2A is a perspective view of a fine needle aspiration device according to one embodiment of the present disclosure;

FIG. 2B is a partial cross-sectional view of an actuator subassembly according to one embodiment;

FIG. 2C is a side elevation view of the actuator subassembly of FIG. 2B in a first configuration;

FIG. 2D is a side elevation view of the actuator subassembly of FIG. 2B in a second configuration;

FIG. 2E is a partial side elevation view of the actuator subassembly of FIG. 2B showing a controller for a stylet in a first configuration;

FIG. 2F is another partial side elevation view of the actuator subassembly of FIG. 2B showing the stylet controller in a second configuration;

FIG. 3A is a partial cross-sectional view of a needle and a stylet according to one embodiment of the present disclosure with the stylet in an extended configuration;

FIG. 3B is a partial cross-sectional view of the needle and the stylet of FIG. 3A with the stylet in a partially retracted configuration;

FIG. 3C is a partial cross-sectional view of the needle and the stylet of FIG. 3A with the stylet in a fully retracted configuration;

FIG. 3D is a schematic view of the needle and stylet of FIG. 3A showing the stylet extension projecting axially from the needle within a target tissue;

FIG. 4A is a partial side elevation view of another stylet of the present disclosure;

FIG. 4B is a cross sectional view of the stylet of FIG. 4A;

FIG. 5 is a partial side elevation view of a stylet and an extension of a stylet according to an embodiment of the present disclosure;

FIG. 6 is yet another partial side elevation view of a stylet and extension of the present disclosure;

FIG. 7 illustrates a stylet and extension according to another embodiment;

FIG. 8A is a partial side elevation view of a stylet of still another embodiment partially retracted in a needle;

FIG. 8B is a partial side elevation view of the stylet of FIG. 8A extended from the needle;

FIG. 9 is a partial side perspective view of a stylet and illustrating an embodiment of a tissue receiving cavity of an embodiment of the present disclosure formed in the stylet;

FIG. 10 illustrates another embodiment of a tissue receiving cavity formed in a stylet of the present disclosure in a partial cross-sectional side view;

FIG. 11 is still another partial cross-sectional side view of a stylet with a tissue receiving cavity of one embodiment of the present disclosure;

FIG. 12 is still another partial cross-sectional side view of a stylet with still another tissue receiving cavity of the present disclosure;

FIG. 13 illustrates a stylet with two tissue receiving cavities of the present disclosure in a partial cross-sectional side view;

FIG. 14 is a partial cross-sectional side view of another tissue receiving cavity formed on a stylet of the present disclosure;

FIG. 15A is a partial side cross-sectional view of a stylet of the present disclosure;

FIG. 15B is an axial cross-sectional view of the stylet of FIG. 15A;

FIG. 16 is a partial perspective view of a stylet partially extended from a needle to an embodiment of the present disclosure, the stylet including a groove that extends axially along the stylet, the groove having sharpened axial edges according;

FIG. 17 illustrates another stylet of the present disclosure with sharpened edges in another partial perspective view;

FIG. 18 is still another partial perspective view of a stylet partially extended from a needle, the stylet including sharpened axial edges according to one embodiment of the present disclosure;

FIG. 19A is a partial perspective view of still another stylet of the present disclosure;

FIG. 19B is a partial side elevation view of the stylet of FIG. 19B partially extended from a needle and illustrating the stylet bending radially outward beyond a diameter of the needle;

FIGS. 20A-20F generally illustrate a method of using a stylet of an embodiment of the present disclosure to collect a sample of a target tissue.

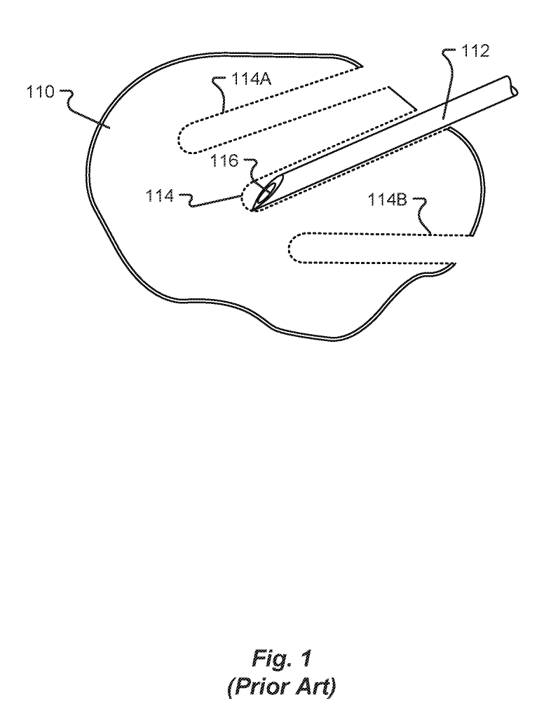

To assist in the understanding of one embodiment of the present disclosure the following list of components and associated numbering found in the drawings is provided:

| Number | Component |
|---|---|
| 110 | Target tissue |
| 111 | Severed tissue |
| 112 | Known needle |
| 114 | Needle pass |
| 204 | FNA device |
| 208 | Actuator subassembly |
| 212 | Needle |
| 214 | Body of needle |
| 216 | Stylet |
| 218 | Stylet proximal portion |
| 219 | Stylet projections |
| 220 | Extrusion |
| 224 | Flexible tubular sheath |
| 228 | Elongated body |
| 232 | Proximal handle portion |
| 236 | Central portion |
| 240 | Distal portion |
| 244 | Attachment portion |
| 248 | First lumen |
| 250 | Proximal end |
| 251 | Actuator for needle |
| 252 | Distal end |
| 253 | Actuator for stylet |
| 254 | Telescoping internal channel |
| 256 | First section |
| 258 | Second section |
| 260 | Third section |
| 264 | Cavity |
| 266 | Lip |
| 268 | Abutment |
| 270 | Proximal opening |
| 272 | Lock mechanisms |
| 274 | Rings |
| 276 | Window |
| 312 | Needle |
| 314 | Longitudinal body of needle |
| 316 | Stylet |
| 320 | Extrusion |
| 378 | Distal end of needle |
| 380 | Second lumen |
| 382 | Tissue receiving opening |
| 383 | Angle of stylet extension |
| 384 | Extension of stylet |
| 385 | Transition zone |
| 386 | Distal end of stylet |
| 388 | Axial distance |
| 390 | Radial distance |
| 416 | Stylet |
| 484 | Extension |
| 485 | Transition zone |
| 486 | Distal end of stylet |
| 487 | Bevel |
| 488 | Stiffness altering features |
| 489 | Opposing sides of stiffness |
| | altering features |
| 491 | Cutting edge |
| 495 | Pocket |
| 516 | Stylet |
| 584 | Extension |
| 585 | Transition zone |
| 586 | Distal end of stylet |
| 588 | Stiffness altering features |
| 590 | Recess |
| 591 | Sharpened edge |
| 616 | Stylet |
| 684 | Extension |
| 685 | Transition zone |
| 686 | Distal end of stylet |
| 688 | Stiffness altering features |
| 690 | Recess |
| 691 | Sharpened edge |
| 716 | Stylet |
| 784 | Extension |
| 785 | Transition zone |
| 786 | Distal end of stylet |
| 790 | Recess |
| 791 | Sharpened edge |
| 812 | Needle |
| 816 | Stylet |
| 878 | Distal end of needle |
| 882 | Needle opening |
| 884 | Extension |
| 886 | Distal end of stylet |
| 916 | Stylet |
| 984 | Extension |
| 985 | Transition zone |
| 986 | Distal end of stylet |
| 987 | Bevel |
| 990 | Recess |
| 991 | Sharpened edge |
| 992 | Tissue receiving cavity |
| 1016 | Stylet |
| 1084 | Extension |
| 1085 | Transition zone |
| 1086 | Distal end of stylet |
| 1087 | Bevel |
| 1090 | Recess |
| 1091 | Sharpened edge |
| 1092 | Tissue receiving cavity |
| 1116 | Stylet |
| 1184 | Extension |
| 1185 | Transition zone |
| 1186 | Distal end of stylet |
| 1187 | Bevel |
| 1190 | Recess |
| 1192 | Tissue receiving cavity |
| 1194 | Projection |
| 1196 | Distal end of projection |
| 1216 | Stylet |
| 1284 | Extension |
| 1285 | Transition zone |
| 1286 | Distal end of stylet |
| 1287 | Bevel |
| 1290 | Recess |
| 1292 | Tissue receiving cavity |
| 1294 | Projection |
| 1296 | Distal end of projection |
| 1316 | Stylet |
| 1384 | Extension |
| 1385 | Transition zone |
| 1386 | Distal end of stylet |
| 1387 | Bevel |
| 1390 | Recess |
| 1392 | Tissue receiving cavity |
| 1416 | Stylet |
| 1484 | Extension |
| 1485 | Transition zone |
| 1486 | Distal end of stylet |
| 1487 | Bevel |
| 1490 | Recess |
| 1492 | Tissue receiving cavity |
| 1496 | Distal end of projection |
| 1516 | Stylet |
| 1584 | Extension |
| 1585 | Transition zone |
| 1586 | Distal end of stylet |
| 1587 | Bevel |
| 1591 | Lateral edge |
| 1598 | Lateral surface |
| 1612 | Needle |
| 1616 | Stylet |
| 1680 | Second lumen |
| 1682 | Needle opening |
| 1684 | Extension |
| 1686 | Distal end of stylet |
| 1687 | Bevel |
| 1689 | Axial groove |
| 1691 | Lateral edge |
| 1698 | Lateral surface |
| 1712 | Needle |
| 1716 | Stylet |
| 1780 | Second lumen |
| 1782 | Needle opening |
| 1784 | Extension |
| 1786 | Distal end of stylet |
| 1789 | Axial groove |
| 1791 | Lateral edge |
| 1812 | Needle |
| 1816 | Stylet |
| 1880 | Second lumen |
| 1882 | Needle opening |
| 1884 | Extension |
| 1886 | Distal end of stylet |
| 1889 | Axial groove |
| 1891 | Lateral edge |
| 1912 | Needle |
| 1916 | Stylet |
| 1980 | Second lumen |
| 1982 | Needle opening |
| 1984 | Extension |
| 1986 | Distal end of stylet |
| 1990 | Recess |
| 1991 | Lateral edge |
| 1992 | Tissue receiving cavity |
| 1999 | Axial extent of needle |
| 2012 | Needle |
| 2016 | Stylet |
| 2078 | Needle distal end |
| 2082 | Needle opening |

### DETAILED DESCRIPTION

Before any embodiments of the disclosure are explained in detail, it is to be understood that this disclosure is not limited in its application to the details of construction and the arrangement of components set forth in the following description or illustrated in the accompanying drawings. The disclosure is capable of other embodiments and of being practiced or of being carried out in various ways. Also, it is to be understood that the phraseology and terminology used herein is for the purpose of description and should not be regarded as limiting.

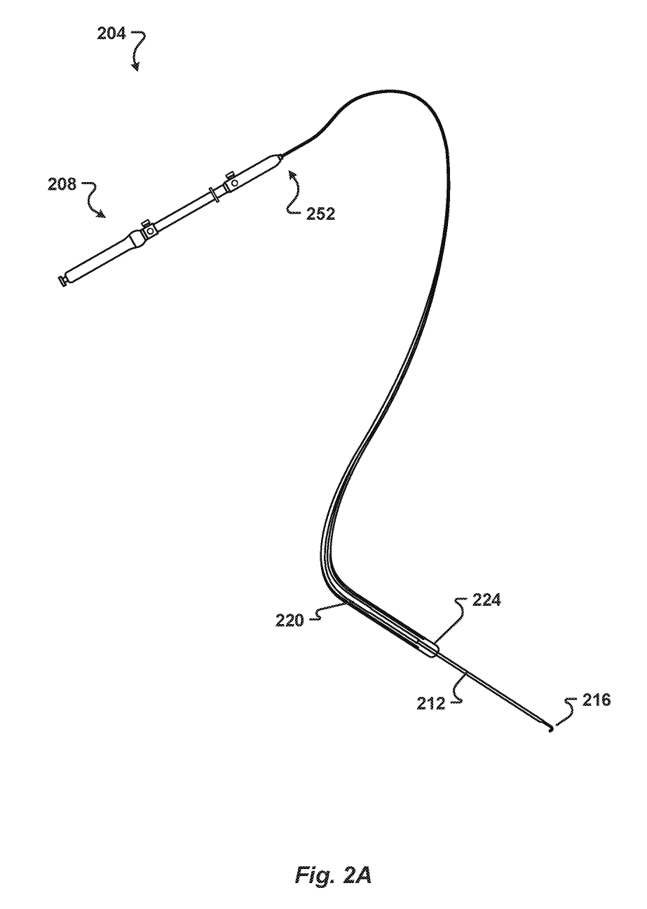

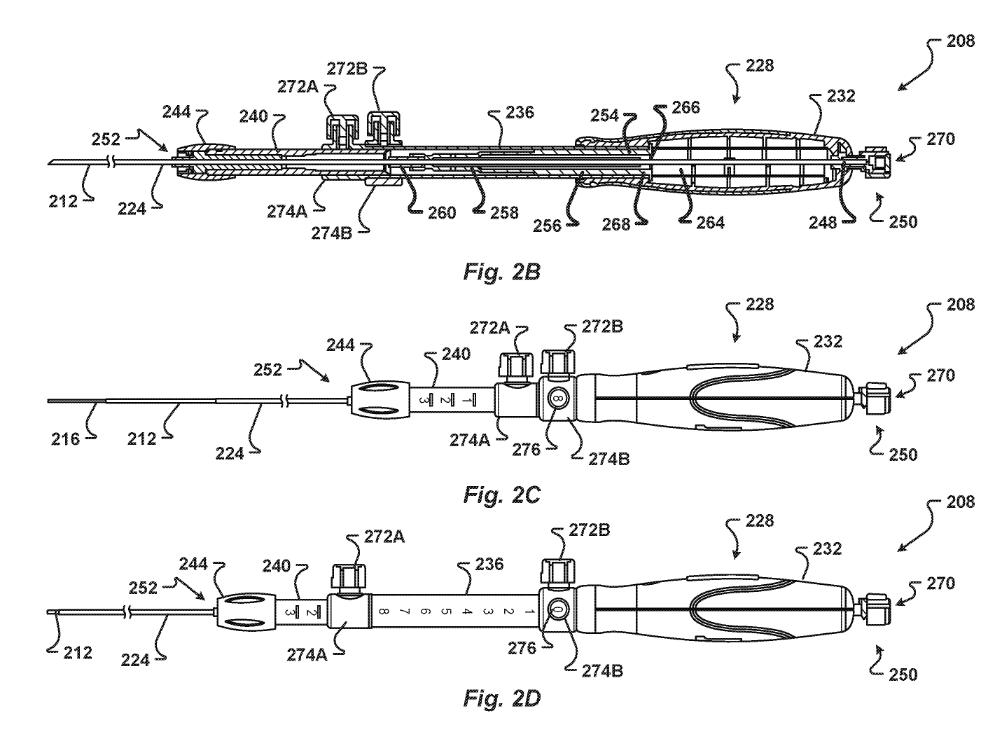

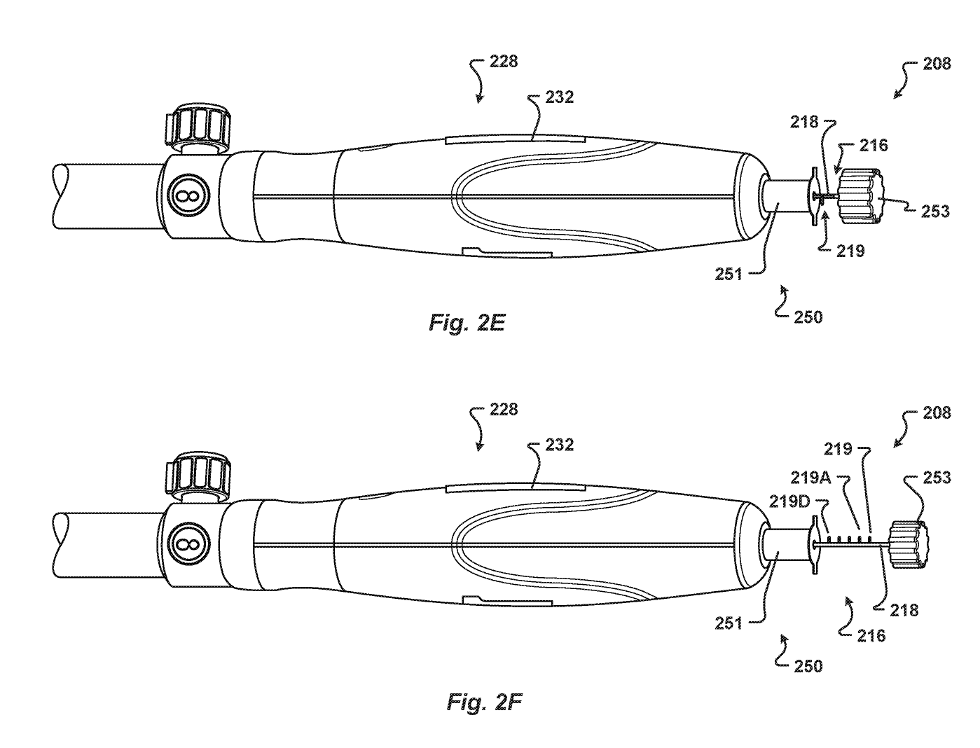

Referring now to FIG. 2A, a device 204 according to one embodiment of the present disclosure for use in EUS-FNA procedure for obtaining tissue samples is illustrated. The device 204 generally includes an actuator subassembly 208, a needle 212, and a stylet 216. The device may also include an extrusion 220 encasing at least a proximal portion of the needle 212. Additionally, a portion of the needle 212 and extrusion 220 are slidably-surrounded by a flexible tubular sheath 224. The sheath 224 is interconnected to the distal end 252 of the actuator subassembly 208. The sheath 224 is sized and shaped for insertion into the working channel of the endoscope (not illustrated) when the actuator subassembly 208 is interconnected thereto. The sheath 224 protects the needle 212 during delivery of the needle 212 to a target tissue within the body of a patient. Once the target tissue has been reached, the actuator subassembly 208 is manipulated to cause a proximal retraction of the sheath 224 to expose a predetermined distal portion of the needle 212.

Referring now to FIG. 2B, a cross-sectional view of actuator subassembly 208 is illustrated. Actuator subassembly 208 generally comprises an elongated body 228 having a proximal handle portion 232, a central portion 236, a distal portion 240, and an attachment portion 244 located at a distal end 252 thereof. Attachment portion 244 permits attachment of actuator subassembly 208 with an endoscope or other device for insertion into a body in an operative configuration. Components of actuator subassembly 208 may be formed of any combination of a polymer, metal or other known materials. The material of actuator subassembly 208 is selected so that, when attached to an endoscope, the elements are permitted to rotate without breaking or cracking.

A first lumen 248 extends through the actuator subassembly 208 from a proximal end 250 to the distal end 252. First lumen 248 is configured to receive needle 212, stylet 216, or another medical device therethrough (e.g., electrodes, knives, pincers, etc.). In example embodiments, first lumen 248 has a substantially circular in cross-section. It is noted, however, that other cross-sectional shapes of first lumen 248 are also envisioned.

Inner walls of a portion of the first lumen 248 extending through the proximal handle portion 232 comprise radial abutments or a treated surface (not shown) to permit a frictional or mechanical engagement with an outer wall of needle 212 inserted therethrough. Needle 212 may also comprise an abutment, a recess or a treated surface (not illustrated) to permit such an engagement. Thus, when inserted through the lumen 248, needle 212 may be moved proximally and distally relative to the proximal handle portion 232 by application of a sufficient proximal or distal force to a proximal end of needle 212. The abutment may also prevent the needle 212 from being rotated relative to proximal handle portion 232. In example embodiments, rotation of needle 212 can only be facilitated by a rotation of proximal handle portion 232. Alternatively, the shapes of any portion of needle 212, stylet 216, or other medical device and a corresponding portion of first lumen 248 may be keyed to one another to prevent relative rotation therebetween.

First lumen 248 also extends through the central portion 236 and distal portion 240 of elongated body 228 of the actuator subassembly 208. However, first lumen 248 is not rotatably fixed to the central and distal body portions 236, 240; i.e., first lumen 248 is rotatable relative to the central portion 236 and the distal portion 240. Thus, actuator subassembly 208 controls rotational movement of needle 212 without the need to rotate the endoscope attached thereto; i.e., rotating proximal handle portion 232 rotates the needle 212 without rotating central portion 236, distal portion 240, or an endoscope interconnected to attachment portion 244.

Central portion 236 and distal portion 240 comprise a telescoping internal channel 254 configured with a first section 256, a second section 258, and a third section 260. First, second, and third sections 256, 258, 260 are configured to be retractable into one another upon retraction of one or both of central and distal portions 236, 240. First lumen 248 extends through the telescoping internal channel 254 and is slidable relative thereto so that retraction and expansion of the telescoping internal channel 254 does not cause proximal or distal movement of the lumen 248. Thus, proximal retraction of the distal portion 240 of elongated body 228 causes the distal portion 240 to be withdrawn into the central portion 236 and retraction of at least third section 260 into second section 258. Similarly, when central portion 236 of the elongated body 228 is retracted into proximal handle portion 232, the outer wall of central portion 236 slides into a cavity 264 within proximal handle portion 232.

First lumen 248 is slidable relative to the telescoping internal channel 254 so that proximal retraction of the central portion 236 and distal portion 240 does not proximally retract the needle 212. Instead, retraction of portions 236, 240 permits a greater portion of the needle 212 to be exposed at a distal end of the device 204, as shown in FIG. 2C. The telescoping internal channel 254 further comprises a lip 266 formed at a proximal end thereof to permit frictional engagement of telescoping internal channel 254 with an abutment 268. The lip 266 and abutment 268 prevent removal of central portion 236 from proximal handle portion 232.

First lumen 248 extends proximally from proximal handle portion 232 by a predetermined distance. An opening 270 into first lumen 248 is provided at proximal end 250 to permit insertion of needle 212, a stylet 216, or other device therethrough. In example embodiments, a tissue collector (not illustrated) may be interconnected to actuator subassembly 208 proximate to opening 270. In this manner, tissue samples that drop from needle 212 or stylet 216 as they are withdrawn from first lumen 248 may be collected. In example embodiments, the tissue collector is a slide.

Central portion 236 further comprises a first mechanism 272A and a second mechanism 272B. These mechanisms 272 are configured to selectively limit a proximal-distal movement of distal portion 240 relative to central portion 236 and movement of central portion 236 relative to proximal handle portion 232. Specifically, first mechanism 272A may be formed as a ring 274A extending around a portion of an outer surface of central portion 236. Ring 274A may be secured to central portion 236 and may be held in place by a friction fit or any other suitable attachment means known in the art. In example embodiments, ring 274A of first mechanism 272A may be permanently secured to a distal end of central portion 236.

Second mechanism 272B associated with central portion 236 is formed substantially similarly to first mechanism 272A. The second mechanism 272B may comprise a ring 274B slidable along a length of central portion 236 to permit advancement of central portion 236 into and out of proximal handle portion 232. Thus, second mechanism 272B may be positioned over a target portion of central portion 236 and tightened to lock the position thereof. In this manner, second mechanism 272B can be positioned so that only a portion of central portion 236 located proximally of second mechanism 272B is retracted into proximal handle portion 232, as shown in FIG. 2C. For example, when a window 276 formed in ring 274B is positioned over the “8” marker at a distal-most position of central portion 236, substantially the entire length of central portion 236 can be drawn proximally into proximal handle portion 232, as shown in FIG. 2C. Similarly, when second mechanism 272B is moved to a proximal-most position along central portion 236 (i.e., so that window 276 is positioned over the “0” marker), central portion 236 is prevented from being retracted into proximal handle portion 232, as illustrated in FIG. 2D.

Referring now to FIG. 2E-2F, in one embodiment, proximal end 250 of the actuator subassembly 208 may include actuators 251, 253 for needle 212 and stylet 216, respectively. The actuators 251, 253 may be used to adjust the relative positions of needle 212 and stylet 216. For example, actuator 253 may be used to advance or withdraw stylet 216 axially with respect to needle 212.

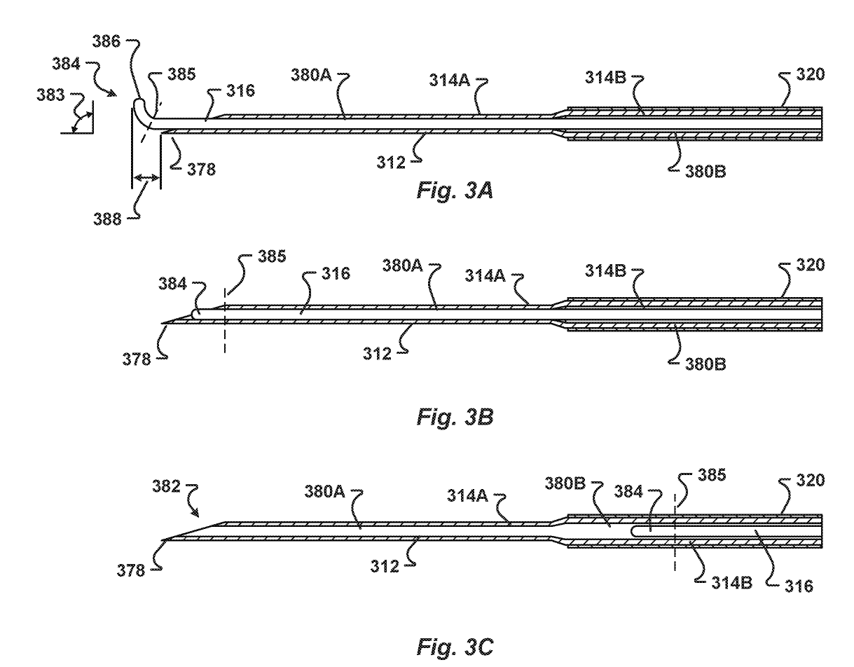

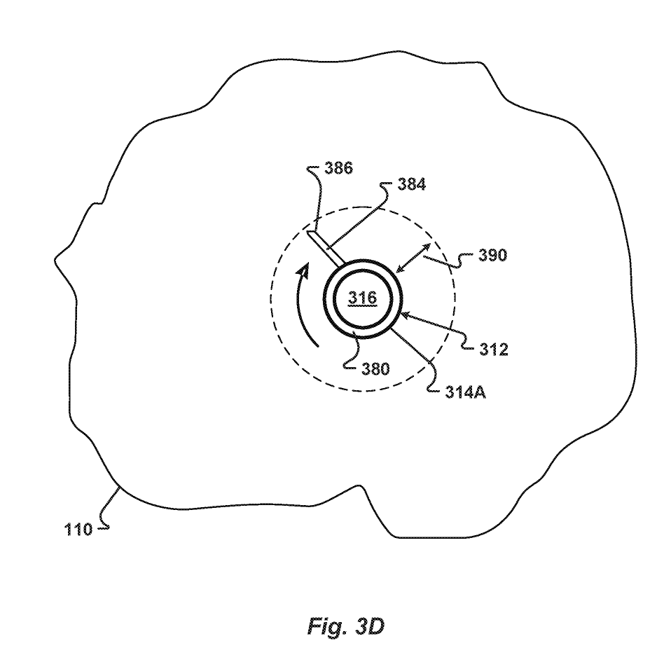

A proximal end 218 of stylet 216 is adapted to provide an indication to the clinician of a relative position of distal end 386 of the stylet with respect to an opening 382 of needle 312 (described in conjunction with FIG. 3). In example embodiments, proximal end 218 includes one or more projections 219 to provide the indication. Projections 219 may be substantially evenly spaced. Alternatively, projections 219 may be irregularly spaced. One or more of the projections may correspond to one or more predetermined positions of stylet distal end 386 in relation to tissue opening 382 of needle 212.

In example embodiments, proximal most projection 219, illustrated in FIG. 2E, is positioned to indicate that stylet distal end 386 is substantially flush with needle opening 382, as generally illustrated in one embodiment in FIG. 3B. Thus, when stylet 216 is arranged substantially as indicated in FIG. 2E, distal end of the stylet 216 substantially seals the opening of needle 212.

A second projection 219A is positioned a predetermined distance from first projection 219 to indicate that distal end 386 of stylet 216 is extended past distal end 378 of needle 212 a predetermined distance, such as illustrated in FIG. 3A. In example embodiments, second projection 219A indicates distal end 378 is extended 1 mm past needle distal end 378. A fifth projection 219D may indicate stylet 216 is fully extended distally, as illustrated in one embodiment shown in FIG. 3A. As an example, fifth projection 219D indicates that stylet distal end 386 is extended 6 mm past needle distal end 378.

The orientation of the projections 219 can also provide an indication of an orientation of the stylet 216 with respect to the needle 212. For example, in FIG. 2E the projection 219 is illustrated projecting radially downward. In contrast, in FIG. 2F the projection 219 is shown projecting radially upward. The orientation of the needle actuator 251 in FIG. 2E is the same as the orientation of the needle actuator in FIG. 2F. Accordingly, the change in the orientation of projection 219 with respect to the orientation of the needle actuator 251 in FIGS. 2E-2F indicates that the stylet 216 has been rotated axially approximately 180° compared to the needle 212.

Other means may be provided to indicate the relative position of distal end 386 of stylet 216 to opening of the needle 212. In one embodiment, one or more notches are provided on proximal end 218 of stylet 216 in substantially the same positions as projections 219. In another embodiment, visual markings provide the indication of the relative position of stylet distal end 386. Projections 219 and/or notches can provide a tactile indication as well as a visual indication to the clinician. Thus, as the clinician moves stylet 216 from the partially retracted configuration (such as illustrated in FIG. 3B) to the extended configuration (illustrated in FIG. 3A), the movement of stylet 216 distally may be accompanied by one or more vibrations or other tactile indications.

Referring now to FIGS. 3A-3C, needle 312 comprises a longitudinal body 314 extending from a proximal end (not shown) to a distal end 378. A second lumen 380 extends through needle 312. Needle 312 is axially flexible along its length so that it may be inserted through the body of the patient to the target area along a tortuous path.

Distal end 378 of needle 312 may include a tapered tip for piercing tissue masses or surfaces and to facilitate penetration of distal end 378 into target tissues. Distal end 378 may be formed with an angled cut. Second lumen 380 extends to a tissue receiving opening 382 at distal end 378 of needle 312. In example embodiments, at least a portion of a surface of needle body 314 surrounding opening 382 is formed as a cutting edge to sever tissue as stylet 316 is retracted into second lumen 380 of needle 312 as will be described in more detail below. Distal end 378 of needle 312 may be formed as an echosonic tip to facilitate viewing under ultrasound imaging.

In example embodiments, longitudinal body 314A of needle 312 at distal end 378 has an exterior diameter that is less than a proximal portion of longitudinal body 314B. The portion of second lumen 380B in longitudinal body 314B has an interior diameter greater than the interior diameter of the portion of second lumen 380A in longitudinal body 314A. However, needle 312 may have an exterior diameter that is substantially constant along its longitudinal body 314. Second lumen 380 may also have a substantially constant interior diameter. The interior diameter of second lumen 380 may remain substantially constant regardless of changes in the exterior diameter of needle body 314.

Longitudinal body 314B may be wrapped or encased by extrusion 320. Extrusion 320 may be extruded from a polymer or other suitable material, such as PFTE (Teflon®); Nylon 12; Pebax 7233; a coil of metal, such as stainless steel; Nitinol; a polymer such as Polyetheretherketon (“PEEK”); or any other suitable material sealed by a shrink wrap or other coating of material.

Needle 312 may be composed of any biocompatible material selected to allow needle 312 to be axially flexible along its length. In example embodiments, needle 312 is formed of a polymer, Nitinol, stainless steel, a chromium cobalt alloy, copper, brass, titanium, aluminum, and combinations thereof. If needle 312 is formed of copper or brass, a coating of a biocompatible material may be applied to the exterior surfaces of the needle and the extension for use within the body of a patient.

Stylet 316 is slidably received within second lumen 380 of needle 312. Stylet 316 is axially flexible along its length so that it may be inserted through, and bend with, second lumen 380. Stylet 316 may be made of any biocompatible material. Optionally, the material of stylet 316 may be selected to provide shape memory. In example embodiments, the stylet is formed of stainless steel; copper; brass; titanium; aluminum; Nitinol; a polymeric material, such as poly-ether-ether ketone, polyamide, poyethersulfone, polyurethane, ether block amide copolymers, polyacetal, polytetrafluorethylene, and/or derivatives thereof; and combinations thereof. If stylet 316 is formed of copper or brass, a coating of a biocompatible material may be applied to the exterior surfaces of the stylet and the extension for use within the body of a patient.

An extension 384 may be formed at distal end 386 of stylet 216. In example embodiments, extension 384 comprises a portion of stylet 316 distal to a transition zone 385. As shown in FIG. 3A, extension 384 is adapted to move radially (or bend) compared to a proximal portion of stylet 316 on a proximate side of transition zone 385. Transition zone 385 indicates an area around which stylet 316 may bend or otherwise move. When stylet 316 is in an extended configuration, transition zone 385 may extend distally beyond distal end 378 of needle 312. In this manner, extension 384 may extend distally beyond distal end 378 of needle 312. Additionally or alternatively, the extension may extend laterally beyond the diameter of longitudinal body 314 of needle 312.

Stylet 316, and its extension 384, may have a shape and a diameter selected to substantially seal the opening 382 when stylet 316 is in the extended configuration or partially retracted configuration. The shape of stylet 316 is also adapted to provide stability and firmness to extension 384 when extension 384 is passed beyond distal end 378 of needle 312 into a target tissue and then rotated.

When the stylet is in a partially retracted configuration, as illustrated in FIG. 3B, extension 384 is substantially within distal end 378 of needle 312. Optionally, stylet extension 384 may substantially seal the opening 382 of needle 312. Thus, stylet 316 can be positioned in the partially retracted configuration during the initial penetration into body tissue to minimize the entry of non-targeted blood and tissue into the needle's second lumen 380 before the target tissue site has been reached. In the partially retracted configuration, extension 384 of stylet 316 returns to a linear configuration substantially parallel to the interior of second lumen 380. Stylet 316 also provides stability and firmness to needle 312 as the needle is passed through the body to a target tissue.

In examples embodiments, the stylet is formed of Nitinol. In related embodiments, at least the extension of the stylet is formed of Nitinol, while the rest of the stylet is formed of another material. For example, the proximate portion of stylet 316 on the proximate side of transition zone 385 may be formed of a metal, such as stainless steel, or a polymer, such as Polyetheretherketon. In related embodiments, stylet 316 of these embodiments may comprise one or more of Nitinol, a metal, and a plastic, in varying proportions. For example, stylet 316 may be formed of a laminate comprising layers of different materials of a variety of thicknesses. Each layer may be of a different material and have a predetermined axial length. The layers may extend generally axially along stylet 316.

Stylets of these embodiments, composed of such combinations of materials can possess unique mechanical properties which may provide benefits to the patient and/or the user of the needle and stylet assemblies. For example, by selecting a transition temperature of the stylet material(s) below room temperature, stylet 316 can have superelastic properties. Thus, extension 384 of stylet 316 may be positioned in the retracted configuration in second lumen 380 of needle 312 for an indefinite period of time without permanent deformation. In the retracted configuration, stylet 316 has a generally straight configuration, illustrated in FIG. 3B. In use, after being inserted into a body of a patient, the temperature of stylet 316 is raised above the critical temperature returning the extension 384 to its original shape, as illustrated in FIG. 3A. The superelastic property, along with the elastic modulus (E) of Nitinol, also permit the transmission of torque from proximal end 350 of device 204 to distal end 386 of extension 384 to generate usable rotation of distal end 386. Alternatively, the superelasticity and ease of shape setting may enable improved cutting or biopsy features of extension 384.

Once distal end 378 of needle 312 has been positioned within a target tissue 110, stylet 316 is moved to the extended configuration. As stylet 316 is moved through distal end 378 of the needle to the extended position, stylet 316 clears any non-targeted tissue that has collected in the opening 382 of needle 312. In one embodiment, extension 384 is positioned beyond distal end 378 of needle 312, as illustrated in FIG. 3A. When transition location 385 extends past distal end 378 of needle 312, extension 384 can move to its original, angled configuration. In example embodiments, extension 384 may extend past the distal end of the needle but remain substantially axially aligned with needle 312.

As illustrated in FIG. 3A, a distal end 386 of extension 384 can extend an axial distance 388 of up to about 6 mm beyond distal end 378 of the needle 312. Stylet distal end 386 may be restricted to extend an axial distance 388 of up to about 3 mm beyond distal end 378. The distance 388 that distal end 386 extends from needle 312 can be controlled by the clinician using device 204. As illustrated in FIG. 3A, an angle 383 between distal end 386 of extension 384 and stylet 316 when transition zone 385 is past distal end 378 of needle 312 may be approximately 90°. However, stylet 316 and extension 384 can be designed to form any desired angle 383, or no angle. Angle 383 between stylet 316 and extension 384 is between about 10° and about 135°. Angle 383 may be between about 70° and about 110°. Angle 383 may be between about 85° and about 95°.

Referring now to FIG. 3D, in example embodiments distal end 386 can extend a radial length 390 of up to about 6 mm laterally beyond the exterior surface of the needle body 314A into target tissue 110. The distal end 386 may also extend about 3 mm in the radial length 390.

After stylet 316 is extended beyond needle opening 382, stylet 316 may be rotated around its longitudinal axis to cut and collect a sample of target tissue 110. When extension 384 is rotated, it cuts and/or scrapes target tissue 110 to collect samples of target tissue. In example embodiments, as stylet 316 is rotated, the extension rotates beyond the circumference of needle body 314A of needle 312 for obtaining tissue samples from more superficially located portions of target tissue 110.

In example embodiments, stylet 316 has a shape that allows it to rotate within the second lumen 380. In these embodiments, stylet 316 is rotated while needle 312 remains substantially stationary. Alternatively, the shapes of any portion of needle 312, stylet 316, or a corresponding portion of second lumen 380 may be keyed to one another to prevent relative rotation therebetween. In these embodiments, after extension 384 of stylet 316 is extended beyond distal end 378 of the needle, both needle 312 and stylet 316 are rotated simultaneously by rotating actuator subassembly 208 of device 204 and/or the endoscope. In other example embodiments, in a first position, stylet 316 can rotate within second lumen 380 while needle 312 remains substantially stationary. In a second position, stylet 316 is keyed to second lumen 380 and stylet 316 and needle 312 rotate simultaneously.

Needle 312 and stylet 316 may include a camming mechanism to rotate stylet 316 relative to needle 312. One of stylet 316 and second lumen 380 may include a spiral groove or track element and the other of stylet 316 and second lumen 380 includes a key element inserted into the groove or track to function as a camming mechanism. Thus, a linear motion of stylet 316 relative to needle 312 is translated into a rotary motion of stylet 316 and extension 384. Further, stylet 316 and needle 312 may include handles at proximate ends thereof. The handles may be connected to one another to facilitate coupling of stylet 316 and needle 312 to prevent inadvertent or unintended relative motion therebetween.

After stylet 316 has been rotated or otherwise manipulated by the clinician while in the extended configuration, stylet 316 is withdrawn into second lumen 380 into a fully retracted configuration as shown in FIG. 3C. When stylet 316 is retracted, extension 384 draws tissue from the target area into opening 382 of needle 312. In example embodiments, when stylet 316 is at least partially retracted into second lumen 380, a suction force is created because distal end 378 of needle 312 is sealed by a target tissue. In this manner, a sample of tissue from target area 110 can be at least partially aspirated into second lumen 380.

Stylet 316 may be completely withdrawn from the opening 270 at the proximal end 250 of the actuator subassembly 208 to collect a sample. Needle 312 can remain in target tissue 110 even when stylet 316 is partially or completely withdrawn from actuator subassembly 208. If an inadequate or non-diagnostic sample of target tissue 110 was collected by stylet 316, a second stylet 316 can be inserted through needle 312 back into the target tissue without creating a second pass through the body of the patient. As will be appreciated, the second stylet may be of the same embodiment as the first stylet. The clinician may also select a second stylet of a different embodiment of this disclosure for reinsertion into target tissue 110 through needle 312.

Although the embodiment of extension 384 illustrated in FIGS. 3A-3C has a curved distal end 386, it should be understood that extension 384 and distal end 386 can have any desired shape adapted to collect samples of target tissue 110. Further, all stylets of this disclosure may be formed with, or without the transition zone. Accordingly, any stylet of this disclosure may be adapted to remain substantially linear when extended from the distal end of the needle. Alternatively, each stylet may be adapted to bend a predetermined amount radially when extended from the needle.

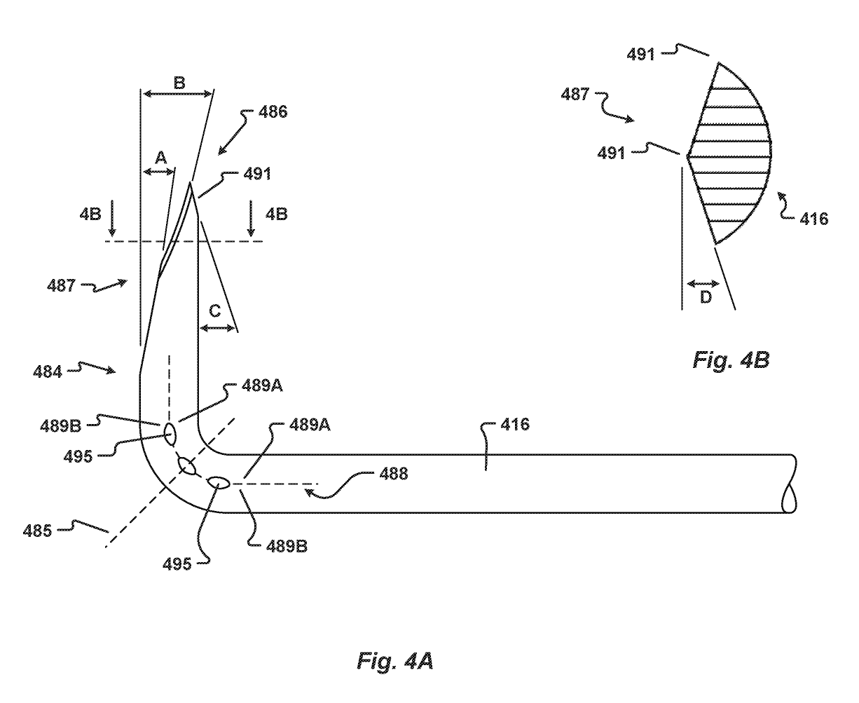

Referring now to FIGS. 4A, 4B, extension 484 has a needle shape with a pointed distal end 486. In addition, edges 491 of extension 484 may be sharpened to cut tissue. A bevel 487 of distal end 486 may face any direction. In example embodiments, bevel 487 faces the proximate end of actuator subassembly 208. In another embodiment, bevel 487 faces the left or right side of stylet 416 such that bevel 487 at least partially faces a direction of rotation of stylet 416. Bevel 487 is adapted to enhance the ability of extension 484 to penetrate target tissue 110.

In example embodiments, bevel 487 incorporates four angular bevel grinds: a primary angle A, a secondary angle B, a back-cut angle C, and tertiary angles D. The primary angle A of the bevel may be from about 10 degrees to about 25 degrees, but is more preferably in the range of about 12 degrees to about 18 degrees. The secondary angle B of bevel 487 may be in the range of about 15 degrees to about 35 degrees, but is more preferably in the range of about 22 degrees to about 28 degrees. Tertiary angle D of the bevel, illustrated in FIG. 4B, may be in the range of about 15 degrees to about 35 degrees, but is more preferably in the range of about 22 degrees to about 28 degrees. Back-cut angle C may be in the range of 15 degrees to about 70 degrees, but is more preferably in the range of about 25 degrees to about 45 degrees. In example embodiments, bevel 487 of extension 484 has an angle substantially the same as the angle of distal end 378 of needle 312 (illustrated in FIG. 3C).

Further, features 488 may be formed in the body of the stylet 416 to alter the bending stiffness of the stylet 416. In example embodiments, features 488 reduce bending stiffness of stylet 416. As illustrated in FIG. 4A, the features 488 comprise slots or cuts that have been formed in the body of stylet 416. Slots or cuts 488 may be formed at any angle with respect to the stylet 416. Slots or cuts 488 may be substantially continuous to form a groove or channel in stylet 416. Slots or cuts 488 may extend partially, or completely, through stylet 416.

In example embodiments, the material of stylet 416 on opposing sides 489 of cuts 488 may be adapted to move at least partially away from each other as distal end 486 of stylet 416 bends. For example, as illustrated in FIG. 4A, the material on opposing sides 489A, 489B of three of the cuts 488 proximate to transition zone 485 have moved apart, forming pockets 495. Pockets 495 may close as opposing sides 489 move together when distal end 486 is retracted proximately into a needle lumen. In this manner, tissue from target area 110 may be trapped, or pinched, at least partially, between opposing sides 489A, 489B as they move together and pockets 495 close.

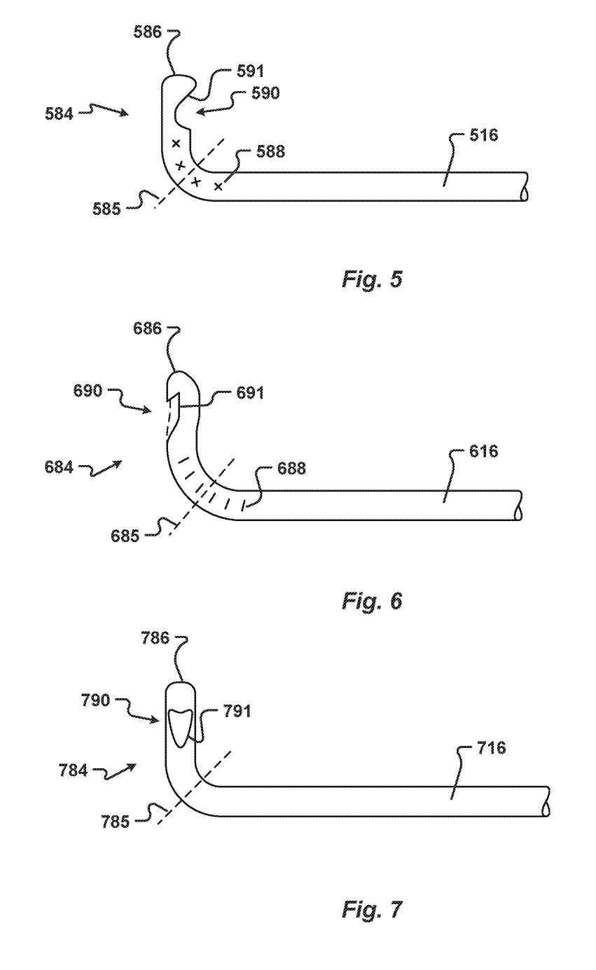

Referring now to FIG. 5, extension 584 has a recess 590. Recess 590 may be of any shape or size. In example embodiments, recess 590 has a scoop or spoon shape. One or more edges 591 of recess 590 may be sharpened to cut and facilitate collection of tissue.

Stylet 516 may also include stiffness altering features 588. Features 588 may comprise a number of cuts that are transverse to the longitudinal axis of stylet 516. Cuts 588 may extend at least partially through stylet 516. Features 588 may include at least some cuts that overlap other cuts. Similar to features 488 illustrated in FIG. 4A, portions of the material of stylet 516 on opposing sides of features 588 may move at least partially away from each other (forming voids or pockets) as distal end 586 of stylet 516 bends.

Recesses may be formed to face any direction on the stylet extension. For example, referring now to FIG. 6, recess 690 may face distally away from actuator subassembly 208. Referring now to FIG. 7, in another embodiment, recess 790 faces a lateral side of stylet 716.

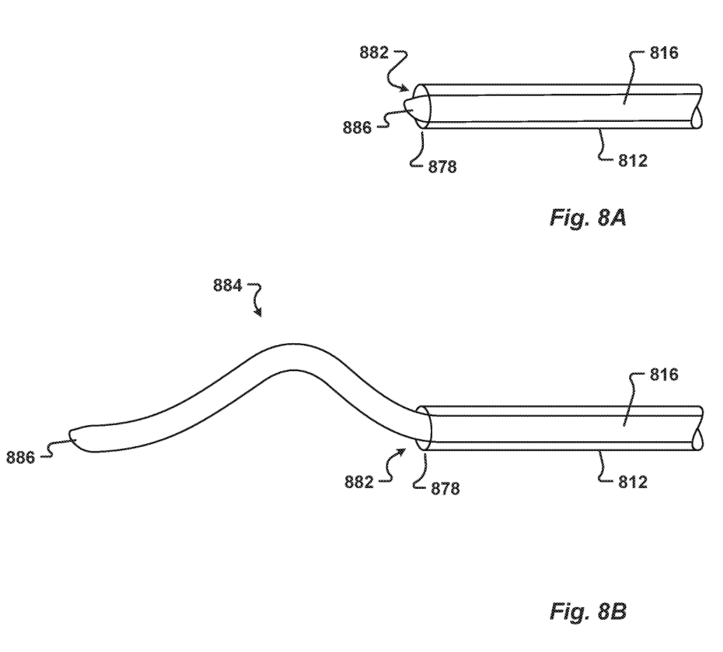

In example embodiments illustrated in FIGS. 8A-8B, extension 884 has an arcuate shape. More specifically, as shown in FIG. 8A, stylet 816 is substantially straight when retracted within the lumen of needle 812. As stylet 816 it advanced distally from distal end 878 beyond opening 882 of needle 812, a portion of extension 884 may bend. A medial portion of extension 884 thus extends beyond the diameter of needle 812. In example embodiments, distal end 886 of stylet 816 is substantially aligned axially with needle 812; i.e., stylet distal end 886 may be positioned generally along a longitudinal axis of needle 812. Cutting edges may be formed on any portion of stylet extension 884.

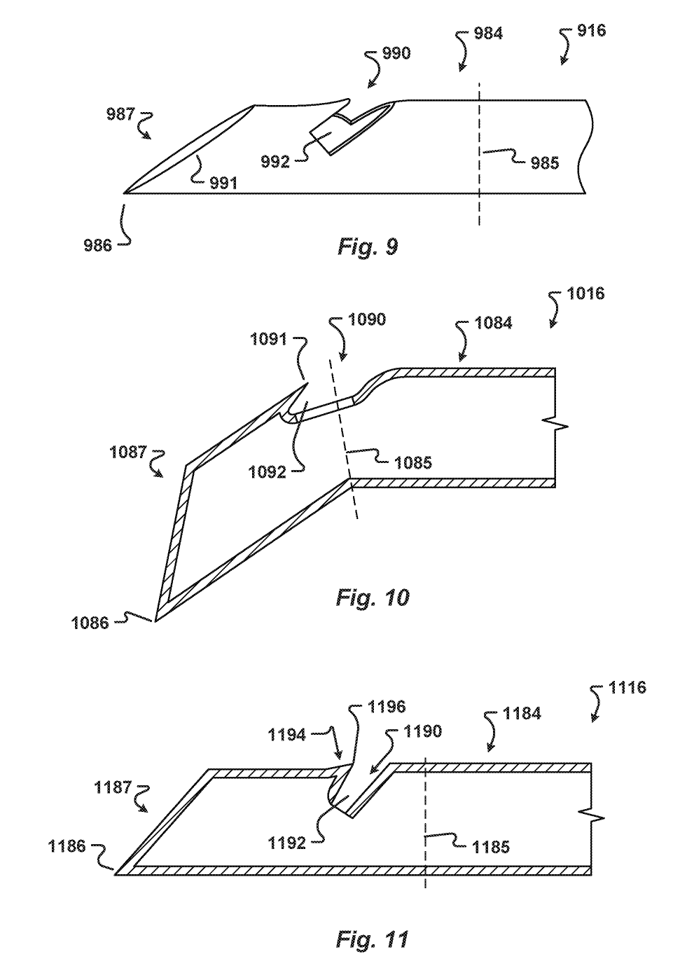

Referring now to FIG. 9, extension 984 of stylet 916, which is illustrated in the generally straight configuration before returning to its original angled configuration, can include a recess 990 with a tissue receiving cavity 992 of any shape or size. Recess 992 is positioned generally transverse relative to a longitudinal axis of extension 984. A circumferential extent of recess 990 is about one-half the circumference of extension 984 of stylet 916. In example embodiments, recess 990 connects to a lumen formed in stylet 916. The lumen extends from recess 990 through stylet 916 to proximal end 250 of actuator subassembly 208. A suction force may be applied to the proximal end of the lumen of stylet 916 to aspirate severed target tissue through recess 990. Transition zone 985 where stylet 916 will bend can be located at any position proximal, distal, or through recess 990.

Referring now to FIG. 10, transition zone 1085 at least partially intersects recess 1090. When stylet 1016 is extended distally from the needle and transitions to its original, angled configuration, as illustrated in FIG. 5B, an opening of recess 1090 may increase in size. As stylet 1016 is moved proximally to the retraced position, extension 1084 bends back to the generally straight configuration and recess 1090 decreases in size, trapping a sample of a target tissue in cavity 1092. Additionally, a sharpened edge 1091 associated with cavity 1092 may move relative to the target tissue as extension 1084 transitions to the straight configuration. Accordingly, sharpened edge 1091 may further sever tissue from the target area of the patient.

As illustrated in FIG. 11, recess 1190 can include a projection 1194 adapted to scrape cellular material from target tissue 110 as extension 1184 is moved relative to a target tissue when stylet 1116 is extended, retracted, and/or rotated. Projection 1194 can have a sharp end 1196 distal to distal end 1186 of stylet 1116. In this manner, as extension 1184 is withdrawn distally into the needle, the sharp end 1196 cuts tissue of target area 119. In example embodiments, the diameter of stylet extension 1184 at sharp end 1196 is less than or equal to the diameter of stylet extension 1184 at a point proximal to recess 1190 so that extension 1184 may be withdrawn into second lumen 380 of a needle. Further, sharp end 1196 may extend laterally beyond the body of extension 1184.

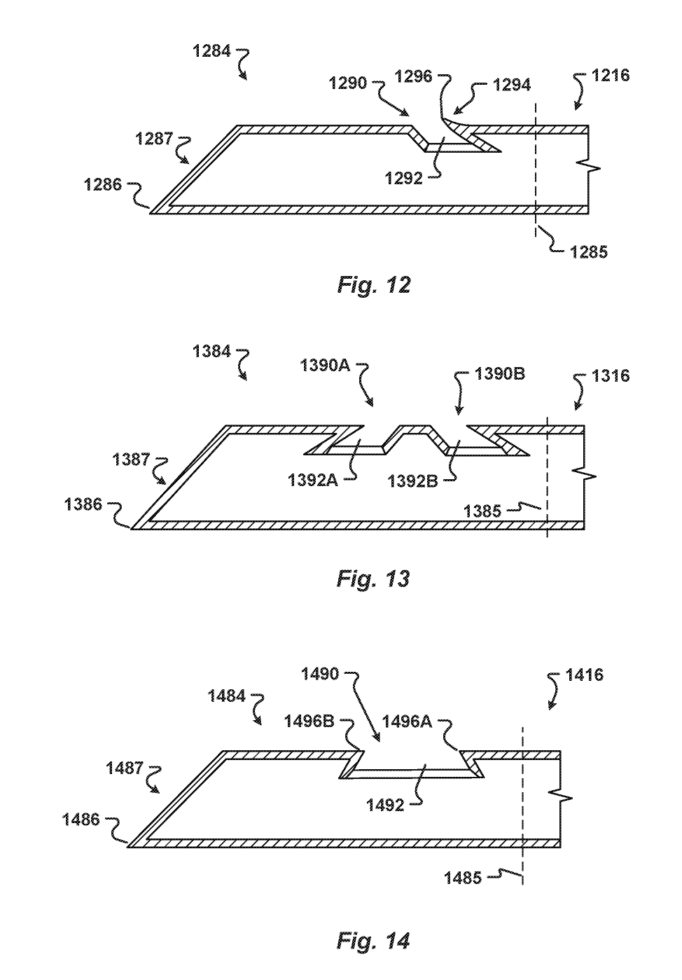

As illustrated in FIG. 12, recess 1290 and cavity 1292 can be positioned at any point on extension 1284 and face any direction. For example, as illustrated in FIG. 12, cavity 1292 faces distal end 1286 of extension 1284 and is positioned distal to transition location 1285. Further, as illustrated in FIG. 13, more than one recess 1390 and cavity 1392 may be formed on an extension 1384 of a stylet 1316. Stylet 1416 may include an extension 1484 that includes a recess 1490 with two sharp ends 1496, as illustrated in FIG. 14.

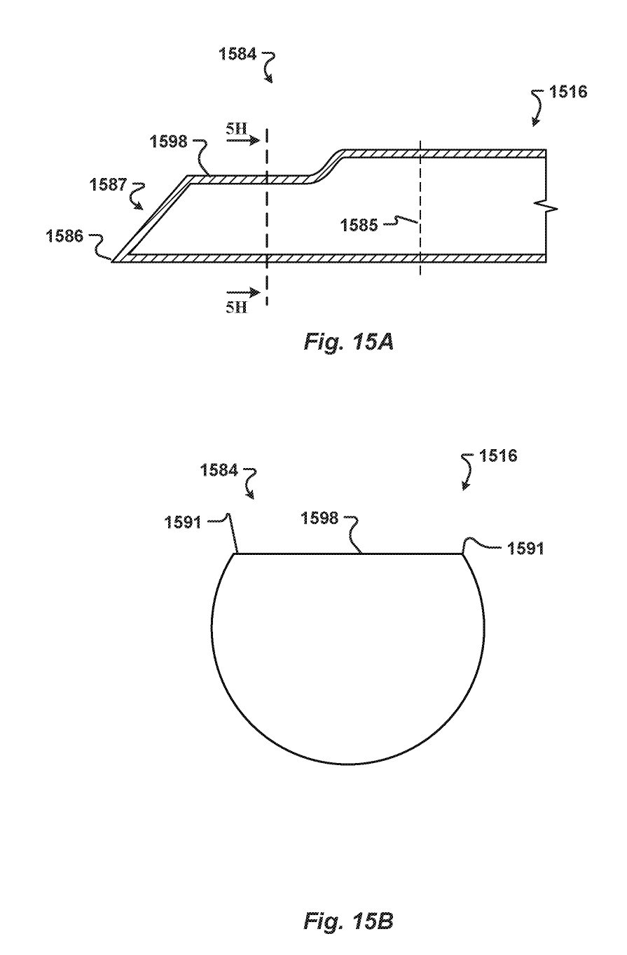

As illustrated in FIGS. 15A-15B, at least a portion of extension 1584 has a lateral surface 1598 that is substantially flat. Lateral surface 1598 forms a chord of the body of extension 1584. Thus, referring now to FIG. 15B, a cross sectional profile of at least a portion of extension 1584 has a generally “D” shape. One or more edges 1591 of lateral surface 1598 may be sharpened to cut tissue when extension 1584 is extended and/or rotated. Stylet 1516 may be formed without transition zone 1585. In this manner, stylet 1516 may extent substantially in line with needle 312 with which stylet 1516 is used.

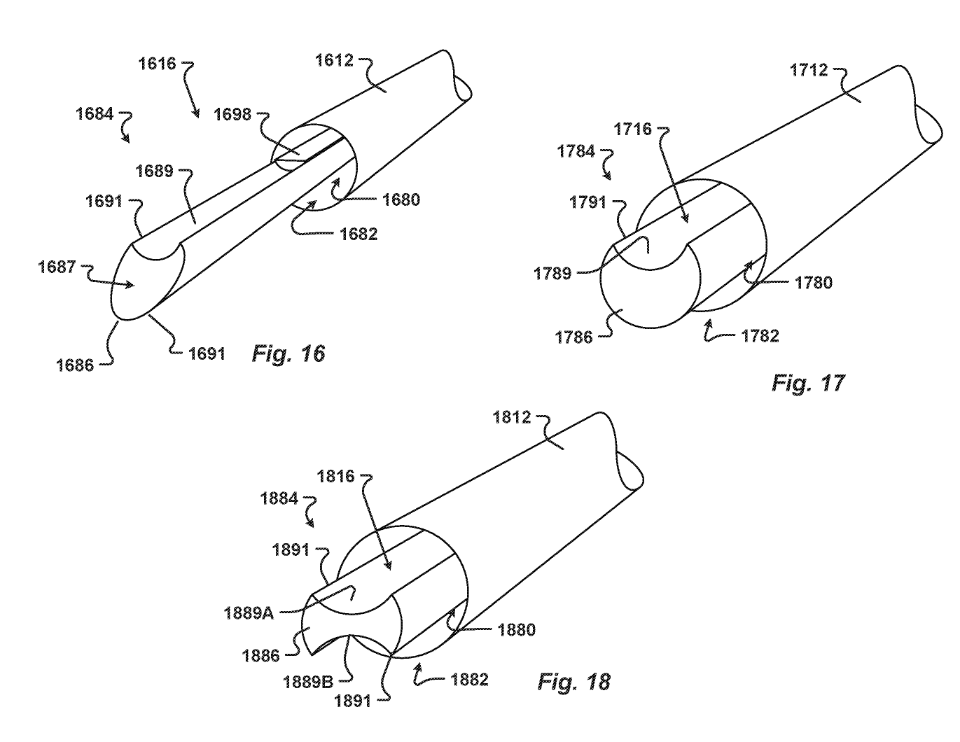

Referring now to FIG. 16, stylet 1616 may include at least one groove 1689. Groove 1689 extends along a predetermined axial length of stylet 1616. Optionally, the groove may run the entire axial length of stylet 1616. In this manner, groove 1689 forms a path through second lumen 1680 of needle 1612. Accordingly, suction applied to the proximate end of needle lumen 1680 may be used to aspirate tissue samples from the target tissue without withdrawing stylet 1616 from the proximal end of needle 1612. Groove 1689 may also end after a predetermined axial length. A lateral surface 1698 may be positioned at the proximal end of groove 1689. Groove 1689 may have any desired shape. For example, groove 1689 may have a generally “U” shaped cross-section. However, other cross-sectional shapes are contemplated. Groove 1689 has a radial depth sufficient to receive and retain tissue samples of targeted tissue.

Lateral edges 1691 of groove 1689 may be sharpened to provide cutting surfaces. In this manner, when stylet 1616 is extended distally from opening 1682 of needle 1612, sharpened lateral edges 1691 as well as bevel 1687 at distal end 1686 of stylet 1616 may cut the target tissue. Further, when stylet 1616 is rotated within a target tissue, lateral edges 1691 may further cut the target tissue.

Referring now to FIG. 17, a stylet 1716 may have an axial groove 1789 that is the same as, or similar to, axial groove 1689 of stylet 1616. However, stylet 1716 is devoid of a bevel at stylet distal end 1786. Distal end 1786 may be substantially perpendicular to the exterior surface of stylet 1716. Stylet 1816 may comprise two or more grooves 1889 as illustrated in FIG. 18. Grooves 1889A, 1889B may have substantially the same, or different, dimensions and cross-sectional shape. Further, although stylet 1816 is illustrated without a bevel at distal end 1886, stylet 1816 may include a beveled end similar to, or the same as, beveled end 1687 of stylet 1616.

Stylets of this disclosure may be adapted to extend distally from the needle lumen in substantially straight alignment with the exterior surface of the needle. For example, stylets 1616, 1716, and 1816, illustrated in FIGS. 16-18, are devoid of transition zones. Accordingly, when stylets 1616, 1716, and 1816 extend distally out of the needle open end, the stylets remain generally straight in axial alignment with their respective needles 1612, 1712, 1812. However, each stylet 1616, 1716, 1816 may optionally have a transition zone as previously described such that the stylet extensions may bend radially a predetermined amount.

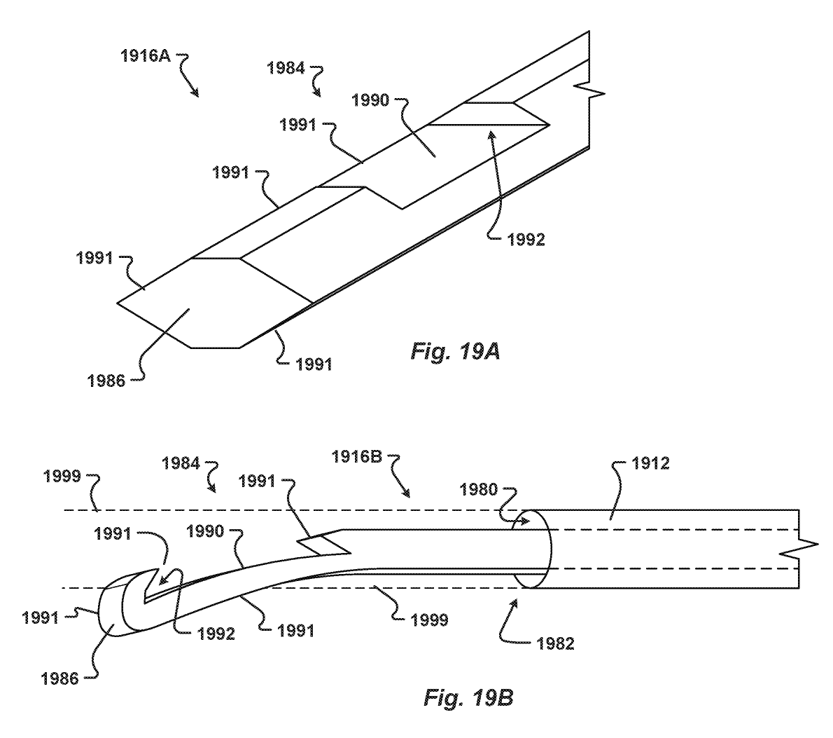

Referring now to FIGS. 19A and 19B, stylets 1916A, 1916B include recess 1990. Recesses 1990 may be of substantially the same size and shape and are similar to the other recesses described in conjunction with FIGS. 9-14. Stylets 1916 include a plurality of lateral edges 1991 that have been sharpened to cut tissue. In example embodiments, all exterior edges 1991 of each stylet 1916 are micro-machined to form a plurality of tissue cutting edges. One or more of stylets 1916 may include a tissue receiving cavity 1992 associated with recess 1990.

Each stylet 1916 is devoid of a transition zone. However, stylets of this disclosure are flexible to facilitate passage over a tortuous route through a patient's body to the site of a target tissue. Accordingly, as stylets 1916 are advanced distally from needle lumen 1980, stylet 1916 may beneficially bend at least partially radially beyond axial boundary 1999 of the exterior of needle 1912. By applying an axial force to stylet 1916, the clinician can control the amount of flexure or bending of distal end 1986. Accordingly, stylet distal end 1986 may extend radially at least partially into portions of the target tissue that have not been disturbed by needle 1912.

In one embodiment, as the clinician advances needle 1912 and stylet 1916, the clinician may use the inherent flexibility of stylet 1916 to guide needle 1912. For example, the clinician may need to avoid a portion of a patient's anatomy to reach the site of the target tissue, and may position stylet distal end 1986 within needle lumen 1980 proximate to needle opening 1982 (similar to the position illustrated in FIG. 3B). The clinician may then advance needle 1912 with stylet 1916 to a predetermined position of the patient's anatomy. At this point, the clinician may extend stylet 1916 distally such that distal end 1986 bends as illustrated in FIG. 19B. The clinician may then maintain the position of stylet 1916 relative to the patient's anatomy while advancing needle 1912 distally over the extended stylet. Thus, the clinician may advance the needle only a path determined by stylet 1916 until needle opening 1982 is proximate to stylet distal end 1986. Accordingly, the clinician can use the flexibility of stylet 1916 to guide needle 1912 through a patient's anatomy.

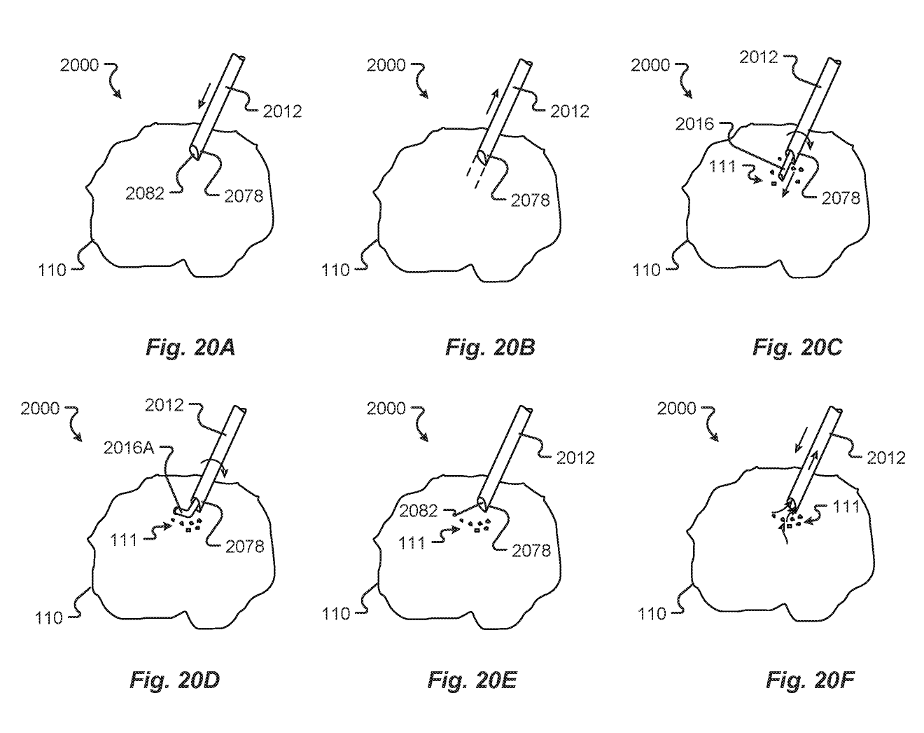

Referring now to FIGS. 20A-20F, a method 2000 of using stylet 2016 to collect a sample of target tissue 110 is generally illustrated. While a general order of operations of the method 2000 are shown in FIGS. 20A-20F, the method can include more or fewer operations or the order of the operations may be arranged differently than those shown in FIGS. 20A-20F. Additionally, although the operations of method 2000 may be described sequentially, many of the operations may in fact be performed in parallel or concurrently. Method 2000 may be performed using any of the devices, needles, and stylets of this disclosure described in conjunction with FIGS. 2-19.

In operation, an endoscope may be attached to the attachment portion 244 of an actuator subassembly 208. Central and distal portions 236, 240 of actuator subassembly 208 are then manipulated by a clinician to a desired orientation. First and second mechanisms 272A and 272B may be tightened to lock actuator subassembly 208 in the desired configuration; i.e., the distal portion 240 may be extended to a length selected such that, when needle 2012 is inserted into first lumen 248 and through the endoscope, needle 2012 is movable between a first position and a deployed position. In the first position, distal end 2078 of needle 2012 may be located within the endoscope lumen (e.g., substantially adjacent a distal end thereof). In the deployed position, needle 2012 projects a desired distance distally beyond a distal end of the endoscope.

Needle 2012 is then inserted through actuator subassembly 208 into the working channel of the endoscope until the proximal end of needle 2012 is locked in position at a proximal end 250 of actuator subassembly 208. Actuator subassembly 208 may be configured so that needle 2012 is in a position with distal tip 2078 thereof received within the endoscope. In addition, stylet 2016 is preferably placed in a partially retracted configuration, such as generally illustrated in FIG. 3B. In the partially retracted configuration, stylet 2016 seals the distal opening of needle 2012 as the needle is inserted through non-targeted tissue to target tissue 110.

Referring now to FIG. 20A, distal end 2078 of needle 2012 is then guided to target tissue 110 within the body of a patient. A clinician determines (e.g., under visual observation via the endoscope) whether distal end 2078 of needle 2012 is in a desired orientation relative to target tissue 110 to be sampled. If distal end 2078 is not in the desired orientation, the clinician may rotate proximal handle portion 132 of the device 208 by a desired angle. The rotation is translated only to first lumen 148 and needle 2012 located therein while central and distal portions 136, 140 of the device 208 and the entire length of the endoscope remain substantially unaffected by the rotation. The clinician may freely rotate the endoscope and/or actuator subassembly 208 by any desired angle until the desired orientation is achieved.

Needle 2012 is then moved distally out of the endoscope to extend distally therefrom by the desired distance, typically under the guidance of an imaging device. Distal end 2078 of needle 2012 may be moved to an observed point in target tissue 110 and then at least partially withdrawn a predetermined distance within target tissue 110; i.e., needle 2012 may be partially withdrawn proximally within target tissue 110 before stylet 2016 is extended, as illustrated in FIG. 20B.

Alternatively, as illustrated in FIG. 20C, stylet 2016 is advanced distally a predetermined distance beyond the needle distal end 2078 without partially withdrawing needle 2012 as described in FIG. 20B. Accordingly, stylet 2016 extends into target tissue that has not been disturbed, and potentially contaminated, by needle 2012. As illustrated in FIG. 20B, a stylet 2016 that is devoid of a transition area may be used with method 2000. Accordingly, stylet 2016 extends generally in line with the axis of needle 2012. However, referring to FIG. 20D, stylet 2016A with a transition area that enables stylet 2016A to bend axially beyond needle 2012 may also be used with method 2000.

The shape and dimensions of stylet 2016, 2016A are adapted to cut and disrupt target tissue 110 and to shear off cellular material 111. Cellular material 111 may be subsequently aspirated into the lumen of needle 2012 after the stylet is at least partially withdrawn from target tissue 110.

In example embodiments, stylet 2016, 2016A may extend a distance of up to about 6 mm beyond the needle distal end 2078. In other example embodiments, the distance is up to about 3 mm. In other example embodiments, the distance is up to about 1 mm. Additionally, distal end of stylet 2016A may extend about 6 mm axially beyond the exterior surface of needle 2012. In other example embodiments, stylet 2016A may extend up to about 3 mm axially In other example embodiments, stylet 2016A extends axially about one-half of the exterior diameter of needle 2012. In other example embodiments, stylet 2016A extends axially about one-fourth the exterior diameter of needle 2012.

Regardless of the type of stylet 2016, 2016A used, after stylet 2016 is extended from needle 2012, stylet 2016, 2016A and, optionally needle 2012, may be rotated axially in either direction. In this manner, cutting surfaces on stylet 2016, 2016A may cut samples 111 from target tissue 110. Further, stylet 2016, 2016A may also be advanced and withdrawn axially to cut target tissue 110. Tissue samples 111 may be collected in (or retained by) the optional cavities and recesses formed in stylet 2016, 2016A.

Referring now to FIG. 20E, after rotating or otherwise moving stylet 2016, 2016A with respect to target tissue 110, stylet 2016, 2016A is retracted proximally into needle 2012 to a fully retracted configuration (illustrated in FIG. 3C). Accordingly, distal opening 2082 of needle 2012 is open to receive tissue 111.

Referring now to FIG. 20F, a suction force is applied to the proximal end of the lumen of needle 2012 to aspirate severed target tissue 111. The aspirated tissue sample is then placed on a slide for analysis. Needle 2012 may be removed from the patient's body. A second stylet may be inserted through the lumen of needle 2012 to target tissue 110. The second stylet may be the same as first stylet 2016, 2016A, or the clinician may select a second stylet with different features than the first stylet 2016, 2016A.

In example embodiments, stylet 2016, 2016A may be completely withdrawn proximally from the lumen of needle 2012. Distal end 2078 of needle 2012 may then be advanced distally into target tissue 110. In this manner, distal end 2078 may be moved into the portion of target tissue 110 that was cut by stylet 2016, 2016A. A suction force is then applied to the proximal end of needle 112 and the needle is simultaneously withdrawn from the target tissue, aspirating severed target tissue 111 into opening 182 of needle 112.

Additionally or alternatively, stylet 2016 may be completely removed from the lumen of needle 2012 to collect target tissue 111 without changing the position of needle 2012 with respect to target tissue 110. In this manner, needle 2012 maintains the path to target tissue 110 such that a second stylet may be inserted through the needle lumen into target tissue 110.

Stylets of this disclosure may be adapted for use with needles of any size, length, or diameter. The stylets may have an exterior diameter adapted to fit within a second lumen of a 22 gauge needle. The stylets may have an exterior diameter adapted to fit within a second lumen 180 of a 19 gauge needle or a 25 gauge needle.

While various embodiments of the invention have been described in detail, it is apparent that modifications and alterations of those embodiments will occur to those skilled in the art. However, it is to be expressly understood that such modifications and alterations are within the scope and spirit of the present invention, as set forth in the following claims. Further, the aspects and embodiments described herein are capable of other embodiments and of being practiced or of being carried out in various ways. In addition, it is to be understood that the phraseology and terminology used herein is for the purpose of description and should not be regarded as limiting. The use of “including,” “comprising,” or “having,” and variations thereof, is meant to encompass the items listed and equivalents thereof, as well as additional items.

1. A stylet for insertion through a lumen of a biopsy needle into a body of a patient comprising:
an elongate body including a distal portion extending longitudinally from a proximal portion; and
an extension formed at the distal portion of the elongate body such that distal advancement of the stylet relative to the needle moves the extension from a retracted configuration in the lumen of the needle to an extended configuration in which the extension extends distally past a distal end of the biopsy needle.

2. The stylet of claim 1, wherein in the extended configuration, the extension projects up to about 6 mm past the distal end of the biopsy needle.

3. The stylet of claim 1, further comprising a tissue receiving cavity formed in the extension.

4. The stylet of claim 3, wherein the tissue receiving cavity has a shape selected from a hook, a spoon, a blade, and a point.

5. The stylet of claim 1, wherein one or more sharp edges are formed on the extension.

6. The stylet of claim 1, wherein the extension is adapted to extend radially past a circumference of a body of the biopsy needle.

7. The stylet of claim 6, wherein at least the extension of the stylet is formed of Nitinol.

8. The stylet of claim 7, wherein:
a critical temperature of the Nitinol is selected to be less than a temperature in an operative environment for the stylet; and
a desired shape for the extension is memorized for temperatures above the critical temperature such that the desired shape of the extension is restored during use when the stylet is in the extended configuration.

9. The stylet of claim 8, wherein the critical temperature is selected to be less than body temperature of the patient.

10. The stylet of claim 6, wherein a distal end of the extension extends up to about 6 mm radially past the circumference of the body of the biopsy needle.

11. The stylet of claim 6, wherein in the extended configuration, an angle between the stylet and the extension is between about 10° and about 135°.

12. The stylet of claim 1, wherein at least a portion of the stylet is formed of one of stainless steel, copper, brass, aluminum, titanium, and combinations thereof.

13. The stylet of claim 1, wherein the stylet has a size and shape adapted to occupy substantially an entire cross-sectional area of a distal opening of the biopsy needle.

14. The stylet of claim 1, wherein the biopsy needle is one of a 19 gauge, a 22 gauge, and a 25 gauge biopsy needle, and wherein the stylet has an exterior diameter adapted to slidably fit within the lumen of the biopsy needle.

15. A method of using a stylet to collect a sample of a target tissue of a patient, comprising:
inserting a biopsy needle through the patient's body into the target tissue;
extending an extension of the stylet distally beyond an opening of the biopsy needle into the target tissue;
moving the extension of the stylet with respect to the target tissue;
withdrawing the stylet at least partially into a lumen of the biopsy needle; and
collecting a sample of target tissue of the patient from at least one of the patient's body, the biopsy needle, the surface of the stylet, and the lumen of the biopsy needle.

16. The method of claim 15, wherein the stylet includes at least one of a sharp edge and a cavity.

17. The method of claim 15, wherein moving the extension with respect to the target tissue comprises at least one of: rotating the stylet axially; and moving the stylet proximally and distally.

18. The method of claim 15, wherein the extension of the stylet is adapted to bend a predetermined amount radially when the extension is moved distally at least partially beyond the opening of the biopsy needle.

19-38. (canceled)

39. The stylet of claim 1, wherein movement of the stylet is controlled by a proximal portion of an actuator subassembly including a first lumen, wherein a portion of the biopsy needle is positioned within the first lumen.

40-67. (canceled)
